(19) United States
(12) Patent Application Publication
(10) Pub. No.: US 2007/0167916 A1
(43) Pub. Date: Jul. 19, 2007

(54) Outer barrel for a syringe

(75) Inventors: Chien-Yuan Lee, Taipei County (TW); Chih-Cheng Kao, Taipei (TW)

Correspondence Address: COOPER & DUNHAM, LLP, 1185 AVENUE OF THE AMERICAS, NEW YORK, NY, 10036, US

(73) Assignee: Linfair Engineering (HK) Co., Ltd.

(21) Appl. No.: 11/331,908

(22) Filed: Jan. 16, 2006

(51) Int. Cl. A61M 5/00

(52) U.S. Cl. 604/187

(57) ABSTRACT

An outer barrel for a syringe having a needle hub mounted on a front end of an inner barrel has at least two halves combined with each other. Each half has a pawl formed at a rear end of the inner surface of the half and selectively engaging with the needle hub. When the inner barrel is pulled out from the outer barrel, the needle hub with a needle is kept inside the outer barrel after the syringe is retracted from the outer barrel. The at least two halves are able to cover syringes with different sizes provided from different syringe manufacturers, so that the outer barrel is versatile in use.

### BACKGROUND OF THE INVENTION

1. Field of the Invention

The present invention relates to an outer barrel, and more particularly to an outer barrel for a syringe that is composed of at least two halves combinable with each other to keep a needle with a needle hub inside the outer barrel after the needle hub as well as the needle is retracted from the outer barrel.

2. Description of Related Art

Medical wastes cause a lot of problems to environment and people, and more particularly to used syringes. When a person is injured by used syringes, the person may be infected with a fatal disease. To prevent used syringes from injuring any person, safety syringes are wildly used in hospitals or any places where perform medical procedures.

With reference to FIGS. 10 and 11, a conventional safety syringe comprises an inner barrel (30), a plunger (32), a retractor (33), a needle hub (34) and an outer barrel (31).

The inner barrel (30) selectively hold liquid medication and has a front opening, a rear opening, an outside surface and a finger flange (301). The finger flange (301) is formed on the outside surface of the inner barrel (30), extends outward from the rear opening of the inner barrel (30).

The plunger (32) has a front and a stopper (321). The stopper (321) is mounted on the front of the plunger (32) and slidably in the inner barrel (30) through the rear opening. When the plunger (32) is pushed into the inner barrel (30), the stopper (321) pushes medication out of the inner barrel (30) from the front opening.

The retractor (33) is mounted removably on the front opening of the outer barrel (30) and has an outer edge, multiple resilience fingers (331) and a front. The fingers (331) are formed longitudinally on the outer edge around the retractor (33) and extend away from the front of the retractor (33).

The needle hub (34) is mounted on the front of the retractor (33) and has a distal end and a needle. The needle is mounted on and protrudes longitudinally from the distal end of the needle hub (34).

The outer barrel (31) has an inner surface, an annular detent (311), a front opening and a rear opening. The rear opening of the outer barrel (31) allows the inner barrel (30) with needle to be inserted into the outer barrel (31). The front opening of the outer barrel (31) allows the needle to extend out of the outer barrel (31). The annular detent (311) is formed on the inner surface of the outer barrel (31) near the rear opening of the outer barrel (31) and selectively engages the fingers (331) on the retractor (33).

After medication is injected into a patient, the inner barrel (30) is pulled out of the outer barrel (31). The fingers (331) on the retractor (33) engage the annular detent (311) in the outer barrel (31). When the inner barrel (31) and the outer barrel (30) are separated, the retractor (33) with the needle is held inside the outer barrel (31) with the engagement between the annular detent (311) and the fingers (331). The needle cannot be used again and cannot injure anyone when the needle is held inside the outer barrel (31).

However, some safety syringe manufacturers will buy syringes from other manufacturers and then assemble the syringes with the outer barrels (31) together to achieve safety syringes. Sizes of the syringes from different manufacturers are different even through the syringes have a same amount of medication capacity. Because the safety syringe manufacturers need to make multiple types of the outer barrels (31) with different sizes to fit with different syringes from different syringe manufacturers, to assemble and to manufacture a conventional safety syringe is troublesome.

To overcome the shortcomings, the present invention provides an outer barrel for a syringe to obviate or mitigate the aforementioned problems.

### SUMMARY OF THE INVENTION

The primary objective of the present invention is to provide an outer barrel for a safety syringe and that can be fit with different syringes with a same capacity and is versatile in use.

The outer barrel for a syringe having a needle hub mounted on a front end of an inner barrel has at least two halves combined with each other. Each half has a pawl formed at a rear end of the inner surface of the half and selectively engaging with the needle hub. When the inner barrel is pulled out of the outer barrel, the needle hub with a needle is kept inside the outer barrel after the syringe is separated from the outer barrel.

Other objectives, advantages and novel features of the invention will become more apparent from the following detailed description when taken in conjunction with the accompanying drawings.

### BRIEF DESCRIPTION OF THE DRAWINGS

FIG. 1 is an exploded perspective view of a syringe with a first embodiment of an outer barrel in accordance with the present invention;

FIG. 2 is an operational side view in partial section of the syringe with the outer barrel in FIG. 1 showing that the inner barrel of the syringe is held inside of the outer barrel;

FIG. 3 is an operational side view in partial section of the syringe with the outer barrel in FIG. 1 showing that the inner barrel pulls out from the outer barrel;

FIG. 4 is an operational side view in partial section of the syringe with the outer barrel in FIG. 1 showing that the needle hub of the syringe engages with the outer barrel;

FIG. 5 is an operational exploded side view in partial section of the syringe with the outer barrel in FIG. 1 showing that the outer barrel and the inner barrel are separated from each other;

FIG. 6 is a side view in partial cross section of a syringe with a second embodiment of an outer barrel in accordance with the present invention showing that the inner barrel is held inside the outer barrel;

FIG. 7 is an exploded perspective view of a syringe with a third embodiment of an outer barrel in accordance with the present invention;

FIG. 8 is an exploded perspective view of a syringe with a fourth embodiment of an outer barrel in accordance with the present invention;

FIG. 9 is an exploded perspective view of a syringe with a fifth embodiment of an outer barrel in accordance with the present invention;

FIG. 10 is an exploded perspective view of a conventional safety syringe with an outer barrel in accordance with the prior art; and

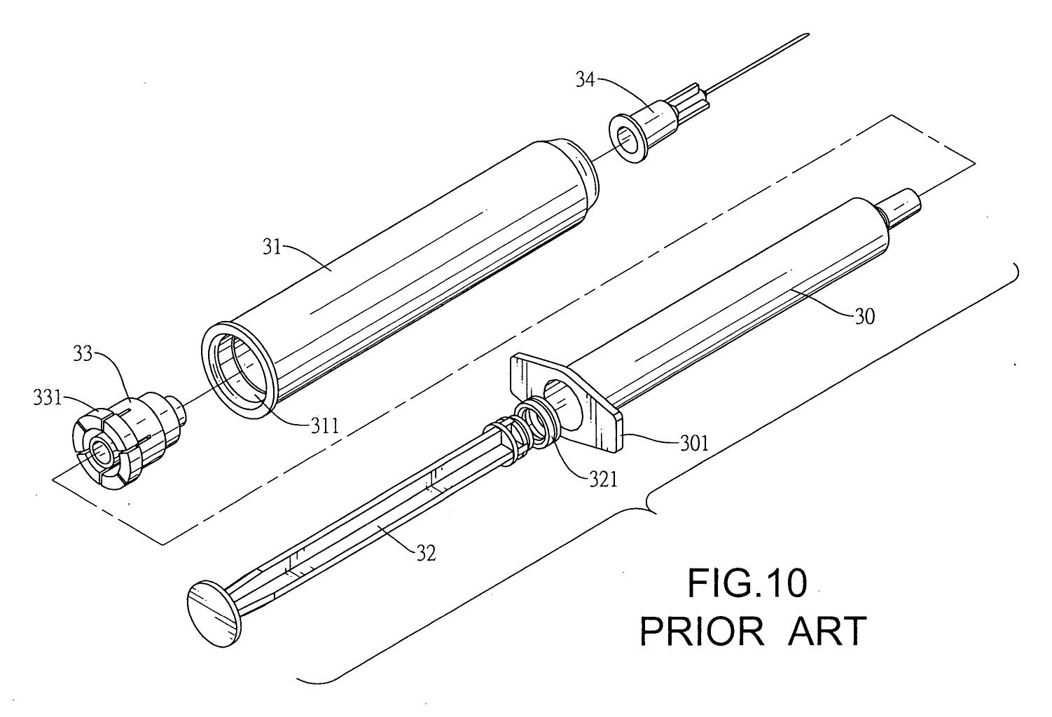

FIG. 11 is a side view in partial section of the safety syringe in FIG. 10 showing that the retractor engages with the outer barrel.

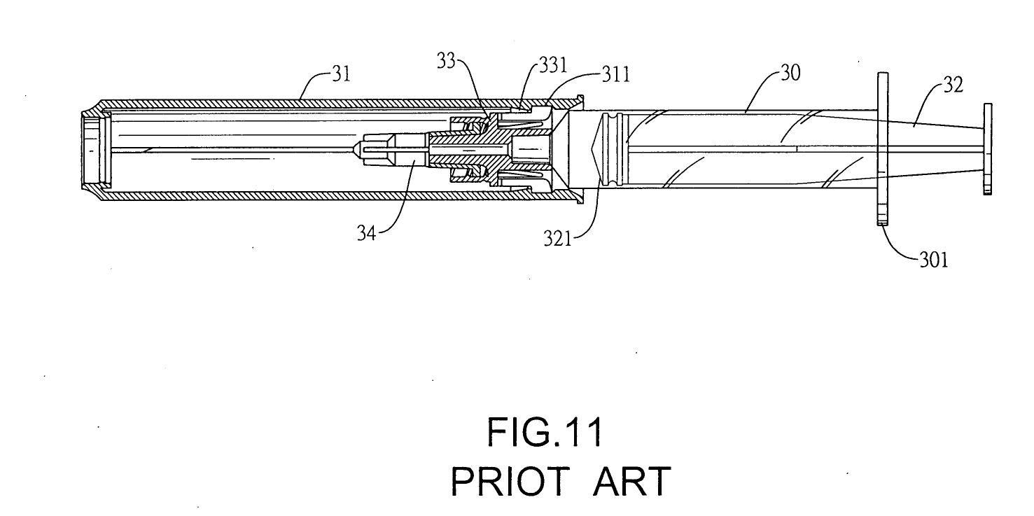

### DETAILED DESCRIPTION OF THE PREFERRED EMBODIMENT

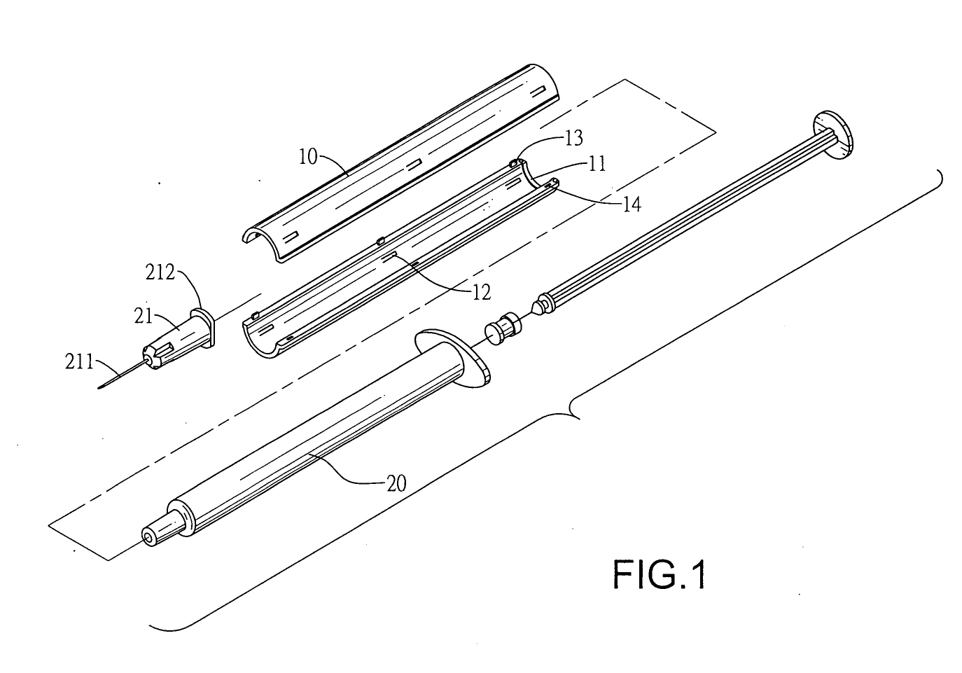

With reference to FIGS. 1, 6, 7, 8 and 9, a syringe comprises an inner barrel (20)(20A)(20B), a plunger and a needle hub (21)(21A)(21B).

The inner barrel (20)(20A)(20B) has a front opening and a rear opening. The plunger is slidably in the inner barrel (20)(20A)(20B) through the rear opening. The needle hub (21)(21A)(21B) is mounted on the front opening of the inner barrel (20)(20A)(20B) and has a front end, a rear end, a needle (211)(211A)(211B) and an engaging device (212)(212A)(22B). The needle (211)(211A)(211B) is mounted on the front end of the needle hub (21)(21A)(21B).

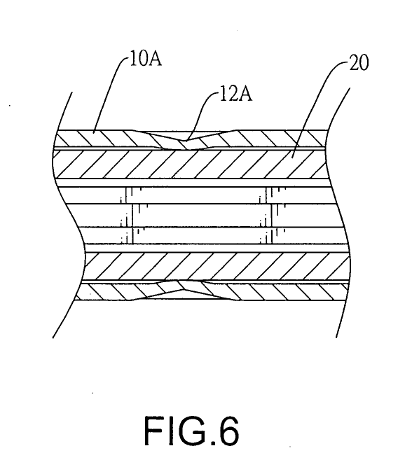

With further reference to FIGS. 1 and 6, in a first embodiment and a second embodiment of the syringe, the engaging device (212) is an annular shoulder. The annular shoulder is formed at and radially extends from rear end of the needle hub (21).

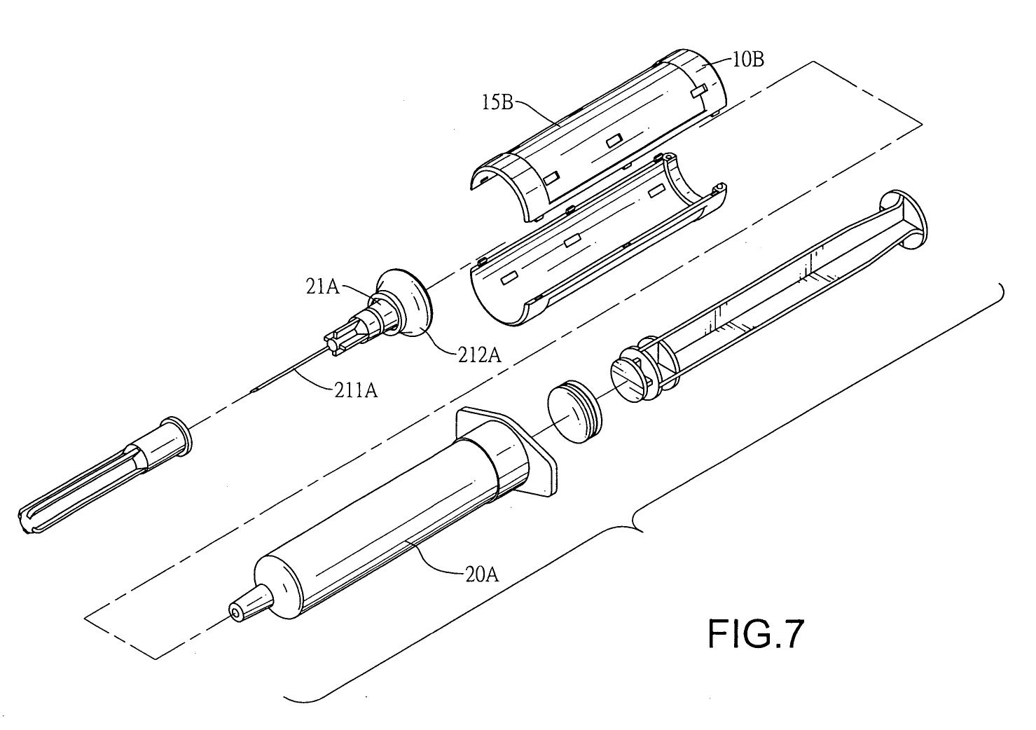

With further reference to FIG. 7, in a third embodiment of the syringe, the engaging device (212A) is a cone-shaped seat. The cone-shaped seat is formed at and axially extends from the rear end of the needle hub (21A).

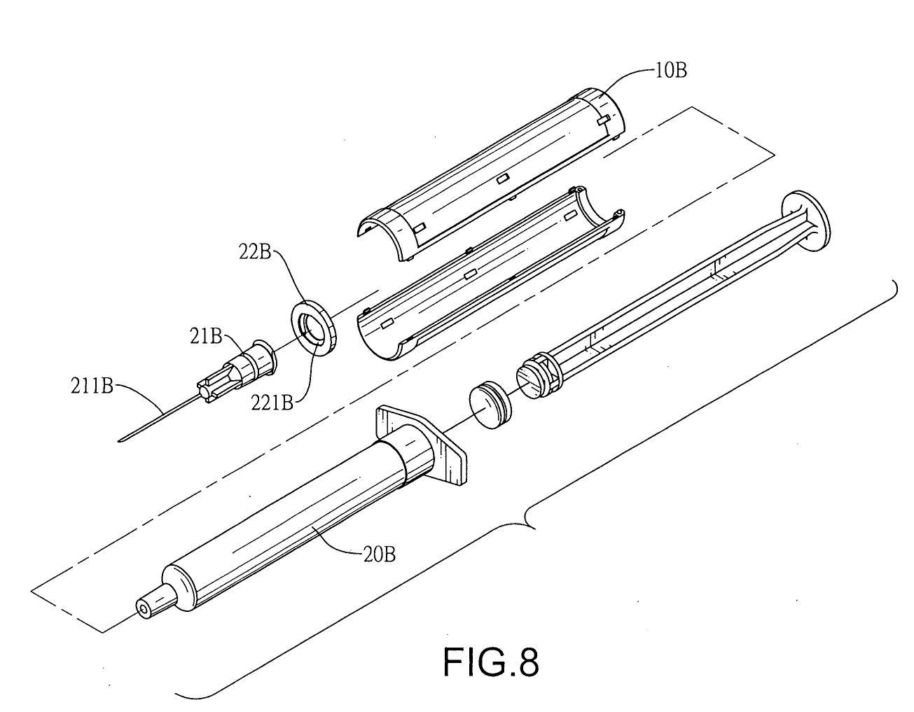

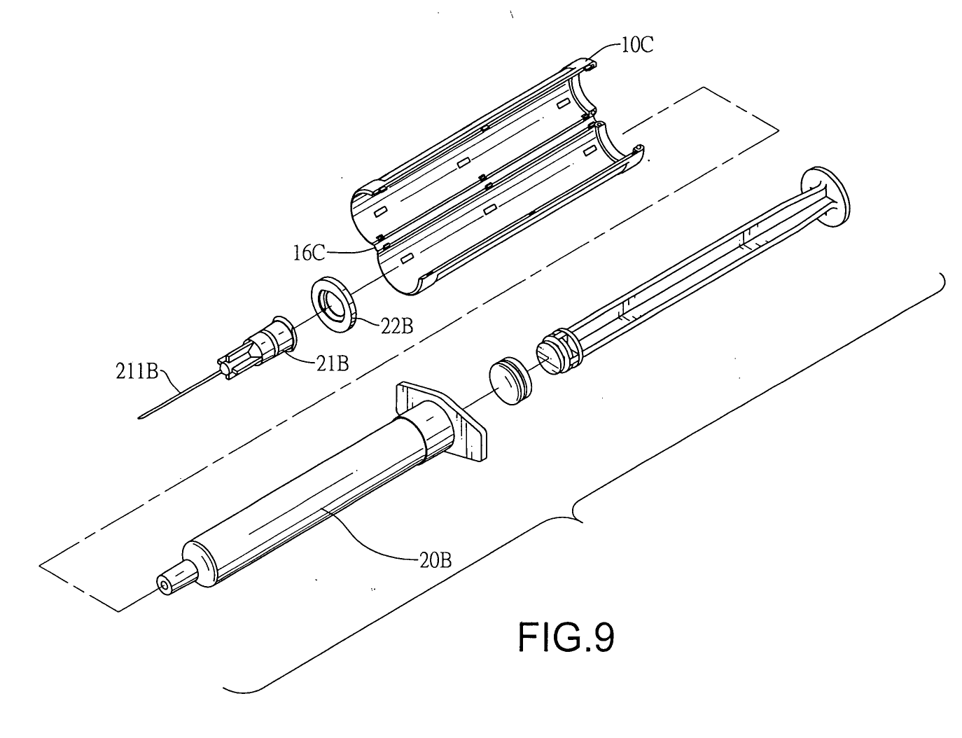

With further reference to FIG. 8 and 9, in a fourth embodiment and a fifth embodiment of the syringe, the engaging device (22B) is a ring and has a central hole (221B). The central hole (221B) is formed at the engaging device (22B) and allow the rear end of the needle hub (21B) to mount on and extend through the central hole (221B).

An outer barrel for a syringe in accordance with the present invention comprises at least two halves (10)(10A)(10B)(10C). Each half (10)(10A)(10B)(10C) has an inner surface, an outer surface, two sides, a pawl (11), multiple stoppers (12), multiple engaging bosses (13), multiple engaging recesses (14), an optional outer recess (15B) and an optional connecting member (16C).

The inner surface of each half (10)(10A)(10B)(10C) has a rear end.

The pawl (11) is formed at and extends inward from the rear end of the inner surface of each half (10)(10A)(10B)(10C).

The engaging bosses (13) and the engaging recesses (14) are formed at two sides of each half (10)(10A)(10B)(10C) and correspond respectively to the engaging recesses (14) and the engaging bosses in the other half (10)(10A)(10B)(10C). When each engaging boss (13) selectively engages with the corresponding engaging recess (14), the at least two halves (10)(10A)(10B)(10C) is combined to an outer barrel that is able to attach to the inner barrel (20)(20A)(20B) of the syringe. The at least two pawls (11) are combined to an annular flange that is able to engage with the engaging device (212)(212A)(22B) of the syringe so that the needle (211)(211A)(211B) is able to be held inside of the outer barrel.

With further reference to FIG. 1, in a first embodiment of the outer barrel for a syringe, the stoppers (12) are resilient flakes and longitudinally and separately formed at the inner surface of each half (10). When the at least two halves (10) are combined to an outer barrel, the stoppers (12) abut with the outer surface of the inner barrel (20) of the syringe.

The stoppers (12) next to the at least two pawls (11) are the strongest and shaped like detents. When the needle hub (21) with the needle (211) is pulled into a space between the at least two pawls (11) and the strongest stoppers (12), the needle hub (21) is kept between the two pawls (11) and the strongest stoppers (12) by the strongest stoppers (12).

With further reference to FIG. 6, in a second embodiment of the outer barrel for a syringe, the stoppers (12A) are protrusions and longitudinally and separately formed at the inner surface of each halve (10A).

With further reference to FIGS. 7 and 8, in a third embodiment and fourth embodiment of the outer barrel for a syringe, the outer recess (15B) is formed at out surface of each half (10B). The outer recesses (15B) reduce the amount of material for forming the halves (10B).

With further reference to FIG. 9, in fifth embodiment of the outer barrel for a syringe, the connecting member (16C) is mounted between and connect sides of two halves (10C), so that when the halves (10C) combine into an outer barrel, the connecting member (16C) is formed and joined between two halves (10C).

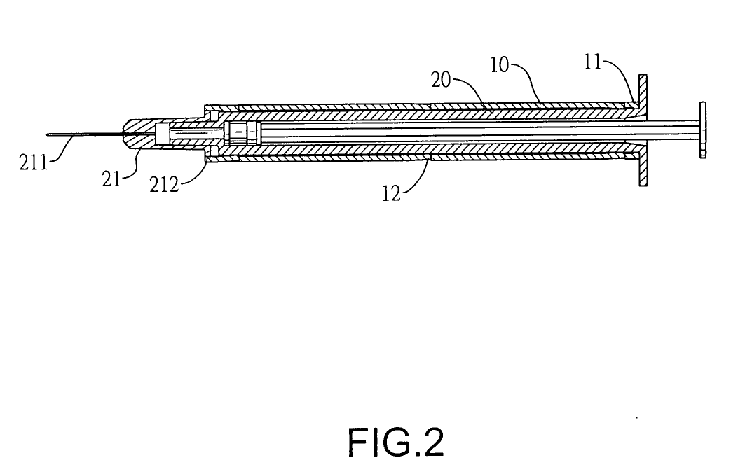

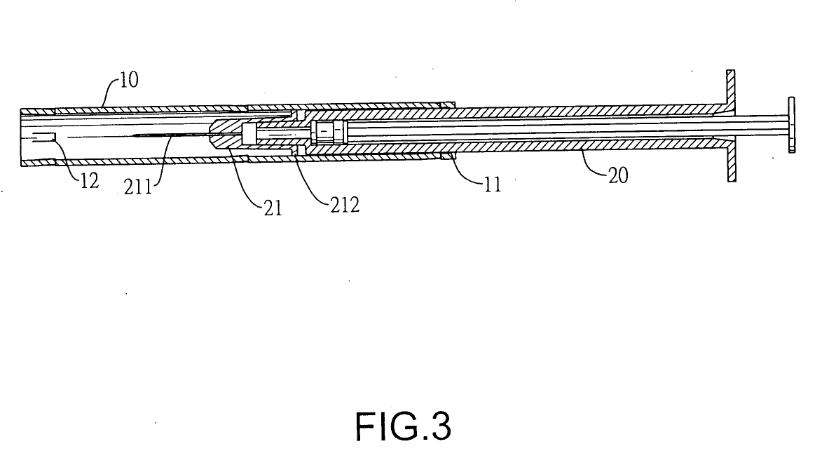

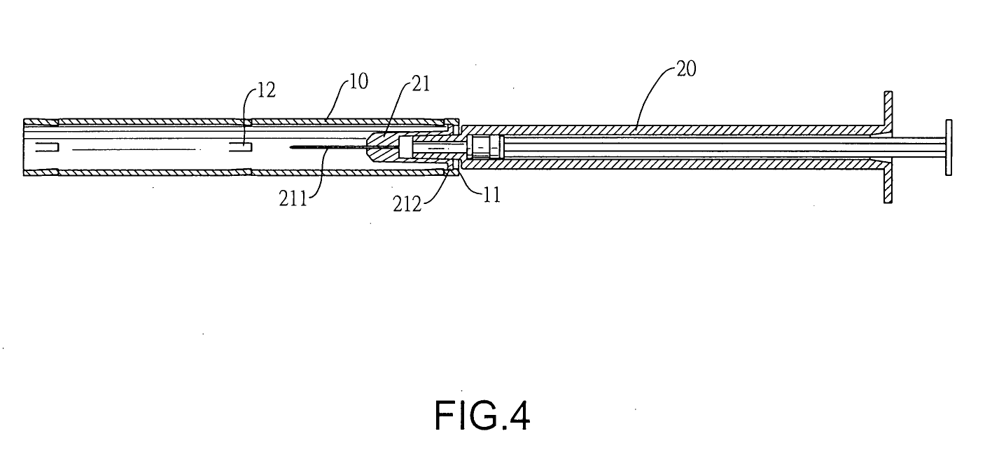

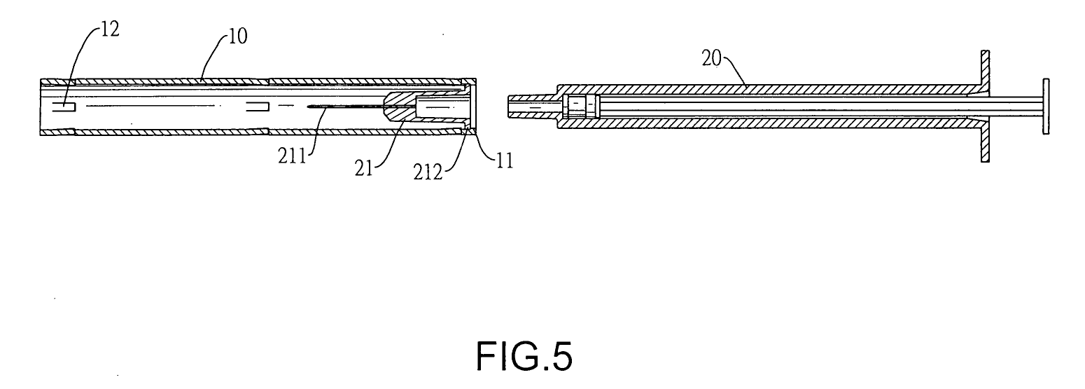

With further reference to FIGS. 2, 3, 4 and 5, after medication is injected into a patient, the inner barrel (20) is pulled out of the outer barrel. The engaging device (212) on the needle hub (21) engages with the annular flange that is formed by the at least two pawls (11) when the at least two halves (10) are combined together. When the inner barrel (20) and the outer barrel are separated, the needle hub (21) with the needle (211) is held inside the outer barrel with the engagement between the engaging device (212) and the annular flange. The needle (211) cannot be used again and cannot injure anyone when the needle (211) is held inside the outer barrel.

Because the stoppers (12)(12A) abut with the outer surface of the inner barrel (20)(20A)(20B) of the syringe so that the halves (10)(10A)(10B)(10C) are able to hold different syringe with different sizes provided from different syringe manufacturers, to assemble safety syringes with different syringes with a same capacity is easy convenient. Therefore, the safety syringe can be easy to manufacture by combining a conventional syringe with at least two halves (10)(10A)(10B)(10C) together.

Even though numerous characteristics and advantages of the present invention have been set forth in the foregoing description together with details of the structure and function of the invention, the disclosure is illustrative only. Changes may be made in detail especially in matters of shape, size, and arrangement of parts within the principles of the invention to the full extent indicated by the broad general meaning of the terms in which the appended claims are expressed.

What is claimed is:

1. An outer barrel for a syringe, the outer barrel comprising:
at least two halves combined with each other, and each half having an inner surface;
an outer surface having a rear end;
two sides;
multiple stoppers longitudinally and separately formed at the inner surface of the half; and
a pawl formed at the rear end of the inner surface of the half and extending inward from the rear end of the inner surface of the half.

2. The outer barrel as claimed in claim 1, wherein each half further has multiple engaging bosses and multiple engaging recesses formed at two sides of the half and corresponding respectively to the engaging bosses of the other half such that after the engaging bosses from one half are received in the engaging recesses of the other half, the outer barrel is formed.

3. The outer barrel as claimed in claim 2, wherein the stoppers are resilient flakes.

4. The outer barrel as claimed in claim 2, wherein the stoppers are protrusions.

5. The outer barrel as claimed in claim 3, wherein the outer barrel further has a connecting member formed and joined between the at least two halves.

6. The outer barrel as claimed in claim 4, wherein the outer barrel further has a connecting member formed and joined between the at least two halves.

7. The outer barrel as claimed in claim 5, wherein each half has an outer recess formed on the outer surface thereof.

8. The outer barrel as claimed in claim 6, wherein each half has an outer recess formed on the outer surface thereof.
